(19) United States
(12) Patent Application Publication
(10) Pub. No.: US 2009/0234445 A1
(43) Pub. Date: Sep. 17, 2009

(54) Catheter device and associated Medical examination and treatment apparatus as well as associated method

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 12/381,512

(22) Filed: Mar. 12, 2009

(30) Foreign Application Priority Data
Mar. 12, 2008 (DE) 102008 013 8588.4

(51) Int. Cl. A61F 2/24; A61B 5/05

(52) U.S. Cl. 623/2.11; 600/424

(57) ABSTRACT

A catheter device for treating a cardiac valve disease, which catheter device has a flexible catheter sheath enclosing a catheter cavity and a catheter-specific module disposed at the proximal end close to the catheter tip for the purpose of transporting and positioning a cardiac valve implant and/or for surgically modeling a cardiac valve, is to be embodied in such a way that the risk associated with a cardiac valve intervention is reduced further in comparison with concepts known and practiced in the prior art. Toward that end at least one sensor for imaging is inventively provided in the region of the catheter tip.

### CROSS REFERENCE TO RELATED APPLICATIONS

This application claims priority of German application No. 10 2008 013 858.4 filed Mar. 12, 2008, which is incorporated by reference herein in its entirety.

### FIELD OF THE INVENTION

The invention relates to a catheter device for the implantation and/or modeling of a cardiac valve, said catheter device having a flexible catheter sheath enclosing a catheter cavity and a module for transporting and positioning or, as the case may be, modeling a cardiac valve or a cardiac valve component being arranged at its proximal end close to the catheter tip, with at least one sensor being provided for imaging purposes in the region of the catheter tip.

The invention further relates to a medical examination and treatment apparatus having a catheter device of the aforesaid type. It also relates to a method for a minimally invasive intervention in a cardiac valve.

### BACKGROUND OF THE INVENTION

Up to the 1990s cardiac valve diseases were usually treated by means of open-heart surgical operations. In that case mechanical or biological cardiac valve prostheses were implanted, or the existing valve opening was reshaped. An intervention of said kind is attended by a high risk and a long period of convalescence for the patient. For some years it has been possible to perform implantations of cardiac valve prostheses or the surgical modeling of cardiac valves minimally invasively with the aid of catheters specially designed for that purpose.

With said minimally invasive method, a thin flexible hollow body or catheter is introduced into the bloodstream (vein or artery) starting from the patient's groin or arm and advanced until the proximal end (the end facing toward the body) of the catheter—the catheter tip—reaches the cardiac valve that is to be treated. Depending on the case, and on the type of cardiac valve, the cardiac valve is subsequently surgically modeled or replaced by an artificial cardiac valve implant.

The former typically takes place in the case of the mitral valve, which can mostly be successfully treated by a change in shape. In this case the valve profile is modeled with the aid of a surgical tool which is enclosed by the catheter-based module and which is controlled via the catheter tube (a procedure known as annuloplasty). When it comes to the aorta valve or the pulmonary valve, on the other hand, an artificial cardiac valve implant integrated into a stent is inserted in most cases. The implant is in this case transported and positioned locally by means of a tool which is specially embodied for the purpose and controlled via the catheter tube and which is likewise enclosed by the catheter-based module.

Upon completion of an intervention in the cardiac valve in question the catheter is extracted again via the bloodstream and thus removed. Often a single application of the method is sufficient to achieve a successful and lasting recovery.

A catheter device for introducing an annuloplasty ring into a cardiac valve opening is known from WO 2004/103223 for example.

Even though the minimally invasive approach represents a significant advance compared with an open-heart surgical intervention, a minimally invasive intervention of said type is nonetheless attended by considerable risks for the patient. Catheters have therefore been developed by means of which the risk involved in a cardiac valve intervention can be reduced further compared with concepts known and practiced in the prior art and the probability of a comprehensive and successful treatment can be increased. Catheters of said type are characterized in that at least one imaging sensor is provided in the region of the catheter tip. A catheter of said type is known from EP 1 534 146 B1 or EP 1 749 475 A2 for example.

### SUMMARY OF THE INVENTION

The object of the present invention is to develop a catheter of the aforesaid type further so that a better image quality can be achieved when necessary. A method for a minimally invasive intervention in the heart is also disclosed.

With regard to the device, this object is achieved according to the invention in that the imaging sensor can be displaced relative to the catheter sheath in the longitudinal direction of the catheter device in such a way that it can be moved into an advanced position outside of the catheter sheath. In other words, it is provided to move the sensor out of a “retracted” stop position located close to the catheter-specific module, in a forward direction out of the outer catheter sheath in order thereby to define, relative to a catheter sheath kept constantly stationary, a variably positionable observation point from which the regions lying further forward can be inspected. For that purpose the imaging sensor can be arranged, for example, on an inner catheter that is displaceable relative to the outer catheter sheath and is arranged in its cavity or on an internal part.

The catheter device is advantageously part of a medical examination and treatment apparatus, wherein the imaging sensor is connected via a signal line routed in the catheter cavity to an image processing and playback apparatus located outside the catheter device and transmits image information to said apparatus in real time from the site of an intervention.

The invention proceeds on the basis of the consideration that a great disadvantage of prior art cardiac valve catheters and their handling resides in the fact that said devices are applied in the heart with the aid of external X-ray fluoroscopy (angiography), with the result that the patient and the medical staff are exposed to X-ray radiation during this procedure. A further disadvantage is that the catheter and/or the cardiac valve to be treated are/is relatively poorly visible in the X-ray image, in particular when a low-cost conventional X-ray method having a two-dimensional imaging characteristic is used. Although the cardiac valve region can be visualized more clearly and in higher contrast by injecting contrast agents, there are patients who have an allergic reaction to contrast agents, which can lead to dangerous complications. Due to the limited resolution of the representation in the case of angiographic X-ray fluoroscopy there is therefore the risk that a cardiac valve implant will not be placed correctly or, in the case of a modeling intervention in a cardiac valve, that the same will be incorrectly reshaped and consequently damaged.

In order to avoid such difficulties it is now provided to arrange an imaging element in the front part of a catheter device in order to transmit “live images” therewith from the site of the minimally invasive intervention, i.e. directly from the heart, to an externally set-up playback apparatus, e.g. a computer-controlled visualization system with attached monitor. By means of said imaging element it is possible firstly to track the introduction and feeding-through of the catheter device through the vessels, heart ventricles and cardiac valves, thereby reducing in particular the risk of a “puncture” through the vascular walls. Secondly, this enables the precise positioning of the catheter-specific module, which includes the transport and positioning tools or the surgical modeling tools, relative to the cardiac valve to be treated to be monitored and checked. An application of X-ray radiation, which, in particular in the case of children, is undesirable and possibly damaging in the long term, can therefore be entirely dispensed with.

The imaging sensor is advantageously configured and aligned in such a way that its field of vision covers a spatial region located around the catheter-specific module. In other words, referred to the catheter sheath arranged approximately cylindrically around a central axis, the imaging sensor “looks” essentially radially outward, and depending on the specific arrangement and according to the type and operating principle of the sensor possibly also “through” the catheter-specific module.

In an alternative embodiment it is provided that the field of vision of the imaging sensor covers primarily the spatial region located in front of the catheter tip, i.e. the sensor “looks” forward referred to the insertion direction of the catheter, which is particularly beneficial for monitoring the introduction process and the catheter's advance, e.g. through a cardiac valve.

The imaging sensor optimally combines the two aforementioned possibilities, which is to say that it has a particularly large field of vision both in the radial and in the forward direction. Alternatively, assuming space conditions permit, it is also possible for a plurality of imaging elements or sensors to be provided which cover the different viewing directions.

The imaging sensor is preferably implemented as an (acoustic) ultrasound sensor, as a magnetic resonance sensor or as an optical image sensor.

Imaging using ultrasound (sonography) is carried out according to what is termed the pulse-echo method. An electrical pulse from a high-frequency generator is converted in the sound head of an ultrasound transducer (mostly a piezoelectric crystal, though a silicon-based sensor is also possible) into a sound pulse and emitted. The sound wave is partially or completely scattered or reflected by the inhomogeneities of the tissue structure. A returning echo is converted in the sound head into an electrical signal and subsequently visualized in an attached electronic analysis and display unit, wherein a 2D or 3D scan of the examination region can be taken by means of a mechanical or electronic swiveling of the sensor. Intravascular ultrasound (IVUS) imaging is particularly suitable for imaging deeper-lying tissue layers and vascular structures.

In a second advantageous variant the imaging sensor is what is termed an IVMRI sensor for intravascular magnetic resonance tomography (IVMRI=Intra Vascular Magnetic Resonance Imaging). In (nuclear) magnetic resonance tomography, the magnetic moments (nuclear spins) of the atomic nuclei of the tissue being examined are aligned in an external magnetic field and excited into a gyratory motion (precession) by means of irradiated radio waves, with an electrical magnetic resonance signal being induced as a result of relaxation processes in an associated receiving coil, said signal constituting the basis for the calculation of the image.

The elements generating the magnetic field as well as the transmitting and receiving coils have recently been successfully miniaturized and integrated in an imaging IVMRI sensor in such a way that an intracorporal or, as the case may be, intravascular application of the MRI method (MRI=Magnetic Resonance Imaging) is possible wherein the requisite static magnetic field is advantageously generated or, as the case may be, applied inside the patient's body. A concept of this kind is described e.g. in U.S. Pat. No. 6,600,319.

For this purpose a permanent magnet or an electromagnet for generating a static magnetic field and a coil acting equally as a transmitting and receiving coil are integrated into the IVMRI sensor. The magnet generates field gradients of preferably 2 T/m to 150 T/m in the vicinity of the vessel or organ that is to be examined. In the vicinity, in this context, means up to 20 mm away from the magnet. Depending on the strength of the magnetic field, radio waves in the frequency range from 2 MHz to 250 MHz can be coupled out via the coil for the purpose of exciting the surrounding body tissue. Higher static magnetic field strengths require higher frequencies for the excitation field. The coil advantageously also serves for receiving the associated “response field” from the body tissue. In an alternative embodiment separate transmitting and receiving coils can be provided.

In contrast to conventional MRI systems, the IVMRI sensor and the electronic circuitry and digital analysis units provided for signal conditioning and analysis are advantageously designed in such a way that they can also operate with high local field gradients even in the case of a comparatively inhomogeneous magnetic field and generate corresponding magnetic resonance images. Since under these conditions the received echo signals are influenced in a characteristic manner by the microscopic diffusion of water molecules in the examined tissue, it is usually possible to achieve an excellent visualization and differentiation between different soft parts, e.g. between lipid layers and fibrous tissue. This is of particular relevance, especially in the now provided application area of minimally invasive interventions.

As an alternative to the concept described here, the static magnetic field can also be generated by means of external magnets. In contrast to conventional MRI, however, the dynamic fields, i.e. the radio waves, are advantageously generated intravascularly in the case of this embodiment also, i.e. by means of a number of transmitting and receiving units disposed on the catheter device.

In an alternative or additional embodiment, an optical imaging element can also be provided in the region of the catheter tip of the catheter device. For example, an optical semiconductor detector based on the well-known CMOS technology (CMOS=Complementary Metal Oxide Semiconductor) can be considered suitable for detecting incident light. Like the CCD sensors (CCD=Charge-Coupled Device) known principally from the field of digital photography, a CMOS sensor of the aforesaid type, also known as an “active pixel sensor”, is based on the internal photoelectric effect and besides having a low current consumption also possesses the advantage that it is particularly cheap to manufacture. With this imaging variant, a suitable light source, e.g. an LED (LED=Light Emitting Diode), must be provided in the region of the catheter tip for the purpose of illuminating the examination and treatment region, specifically the respective cardiac valve, which light source can be supplied with electric current via an electrical line routed through the catheter cavity.

In a further embodiment variant the catheter device can also be equipped with an OCT sensor (OCT=Optical Coherence Tomography).

Optical coherence tomography imaging delivers high-resolution images which comparatively accurately reproduce in particular the structures close to the vessel surface. The principle of this method is based on the fact that light, preferably infrared light, supplied by the catheter device via a fiber-optic light guide is beamed into the vessel, the light reflected there being coupled back into the fiber-optic light guide again and routed to an analysis apparatus. In the analysis unit—as in the case of a Michelson interferometer—the interference of the reflected light with the reference light is analyzed in order to generate the image.

Whereas conventional interferometric equipment preferably operates with laser light of a defined wavelength, which light possesses a comparatively great optical coherence length, with the so-called LCI method (LCI=Low Coherence Interferometry) light sources with broadband radiation characteristics (“white light”) and with a comparatively low coherence length of the emitted light are used. Corresponding image sensors which are now provided according to an advantageous embodiment of the invention for use in the catheter device, are described for example in US 2006/0103850.

In an advantageous modification an image sensor can also be provided which is based on the so-called OFDI principle (OFDI=Optical Frequency Domain Imaging). This method is related to OCT, but uses a wider frequency band. The operating principle is described in more detail e.g. in the publication “Optical frequency domain imaging with a rapidly swept laser in the 815-870 nm range”, H. Lim et al., Optics Express 5937, Vol. 14, No. 13.

Finally, the catheter device can also have an imaging sensor which is based on what is termed “near-infrared (NIR) diffuse reflectance spectroscopy”.

Moreover, combinations of at least two optical sensors of the aforementioned type can also be present.

A tabular overview summarizes the strengths and weaknesses of the respective optical imaging methods (from ++=particularly good or suitable, to −−=deficient or unsuitable):

| Comparison of | | | |
|---|---|---|---|
| image sensors | Near resolution | Far resolution | Penetration of blood |
| Optical (CMOS) | + | + | − |
| OCT | ++ | − | −− |
| LCI | + | + | + |
| NIR | − | − | +/− |
| OFDI | ++ | − | + |

Since the solid angle detectable or, as the case may be, to be monitored by means of the respective image sensor is usually limited, it is advantageous in particular in the case of the already mentioned configuration with radial line of sight (in relation to the central axis of the catheter device) if the imaging sensor can be rotated relative to the catheter sheath by way of a driveshaft routed in the catheter cavity. By this means it is possible to obtain a 360° panoramic view without the need to rotate the catheter sheath itself relative to the vessel path.

Alternatively it is also conceivable to dispose a plurality of imaging sensors distributed over the circumference of the catheter sheath and fixed in a stationary position relative to the latter and provide a cyclical data readout from the sensors. With this configuration only a single signal line is therefore required inside the catheter sheath, via which signal line the image data of the different sensors is sent or, as the case may be, polled sequentially in the manner of a serial interface. A small number of signal lines, preferably only a single one, limits the amount of space required inside the catheter sheath and is therefore of advantage in terms of the usability of the mechanical flexibility and pliability of the catheter sheath.

A multiplexer is preferably provided in addition for purposes of a cyclical data readout from the sensors.

By (mechanical or electronic) rotation of the image sensor with simultaneous retraction or advancing it is advantageously possible to generate 3D images or, as the case may be, volume datasets by means of suitable signal conditioning and image calculation methods known in principle from the prior art.

In an advantageous development a number of position sensors or position transmitters are arranged in the region of the catheter tip in order to enable the current position and preferably also the orientation of the catheter tip to be determined. Preferably said sensors consist of several, in particular three, electromagnetic transmitting coils which interact with a number of receiving coils or signal detectors disposed externally, i.e. outside the patient. In an alternative embodiment the roles of the transmitting and receiving units can also be reversed, i.e. the receiving coils are fixed on the catheter side, while the transmitting coils are preferably arranged stationary in space. The position information thus obtained not only facilitates the reliable introduction of the catheter device and its navigation to the target region; it also supports in an advantageous manner the construction of three-dimensional images from a plurality of two-dimensional cross-sectional images. Furthermore, the position data can advantageously be incorporated into the computational correction of motion artifacts and the like.

In a further beneficial embodiment at least one magnetic element can be provided in the region of the catheter tip for the purpose of guiding the catheter device by means of an external magnetic field. With this magnetic navigation, as it is called, the catheter device is therefore controlled and driven by means of an external magnetic field. The respective magnetic element can be a permanent magnet or an electromagnet.

As an alternative to the guiding of the catheter device by means of an external magnetic field, a mechanical means of navigation can be provided. For that purpose suitable mechanical elements, e.g. in the form of pull wires and the like, are advantageously integrated into the catheter device, which mechanical elements permit a temporary mechanical deformation, extension and/or deflection of the catheter or of individual, selectable catheter sections, in particular the catheter tip, by means of external tensile and compressive forces. The mechanical and/or magnetic guiding of the catheter device is preferably performed automatically with the aid of a computer-based control and drive device.

It can additionally be provided to introduce the actual catheter device into the organ that is to be treated through an outer guide catheter. It is then possible, for example, if this is advantageous or useful for diagnostic purposes, to replace a catheter device using IVMRI imaging by a catheter device using optical imaging without the patient being put under stress by a new invasion, that is to say by a change or a movement or other manipulation of the outer guide catheter. The replacement inner catheter also does not have to be painstakingly navigated into the target region first and re-adjusted there. Rather, it is sufficient to insert it as far as a stop position into the cavity of the outer catheter which remains in its previously reached or assumed position in the vessel or in the heart during the procedure.

An advantageous workflow for the deployment of the catheter device having integrated imaging looks for example as follows:
1. Positioning of the patient on the treatment table
2. Possibly preparatory X-ray examination and/or extracorporal ultrasound examination
3. Introduction of the catheter device via venous access port
4. Guiding of the catheter device based on the integrated imaging up to the cardiac valve to be treated (while simultaneously transporting a cardiac valve implant on the catheter-specific module if necessary)
5. Monitoring of the cardiac valve to be treated by means of the integrated imaging of the catheter device,
6. Positioning of the catheter device with the aid of the integrated imaging
7. Positioning of the cardiac valve implant or surgical modeling of the cardiac valve profile with the aid of the catheter-specific module while monitoring in real time by means of the integrated imaging
8. Concluding double-check by means of the integrated imaging
9. Possibly supplementary concluding X-ray review examination and/or extracorporal ultrasound examination
10. Transfer of the patient.

Prior to performing step 3 and the following steps it could be beneficial or necessary to guide a so-called valvuloplastic catheter having an expandable dilatation balloon up to the cardiac valve to be treated and dilate or, as it were, “force open” said valve by expansion of the dilatation balloon. Alternatively the catheter device described here could also be equipped with a dilatation balloon that is reversibly expandable via an expansion means supplied through the catheter tube, by means of which a cardiac valve dilation of this type can be performed—advantageously monitored or assisted by means of the integrated imaging—prior to the implantation of the cardiac valve prosthesis or prior to the annuloplastic intervention.

Depending on the type of imaging and its ability to “penetrate” blood, it can be useful during step no. 5 to no. 8 to flush the region to be observed with a physiological saline solution in order to displace or dilute the blood once briefly or in a briefly pulsed manner in periodic repetition cycles. It can also be helpful to apply a contrast agent at the site under observation, based, for example, on gadolinium in the case of IVMRI imaging, or based on sulfur hexafluoride in the case of ultrasound imaging. The injection is advantageously performed via an injection tube or the like routed in the catheter cavity and having an outlet aperture in the region of the catheter tip.

To sum up, the catheter device described here principally enables medical workflows to be optimized in the case of a minimally invasive intervention in the heart of a living being. Interventions of said kind can be completed with a higher degree of patient safety and at the same time quicker than previously.

### BRIEF DESCRIPTION OF THE DRAWINGS

Various exemplary embodiments of the invention are explained in more detail with reference to a drawing, in which, in a greatly simplified schematic representation in each case:

FIG. 1 shows a medical examination and treatment apparatus having a catheter device represented in longitudinal section,

FIG. 2 to 6 show alternative embodiments of a catheter device,

FIG. 7 shows a detail view of an optical sensor integrated in a catheter device with lateral/radial observation direction,

FIG. 8 shows a detail view of an optical sensor integrated in a catheter device with forward-directed observation direction,

FIG. 9 shows a detail view of a sensor head integrated in a catheter device for OCT or LCI imaging with lateral/radial observation direction,

FIG. 10 shows a detail view of a sensor head integrated in a catheter device for OCT or LCI imaging with forward-directed observation direction,

FIG. 11 shows a detail view of an integrated sensor for IVMRI imaging with lateral/radial observation direction, and

FIG. 12 shows a detail view of an integrated sensor for IVMRI imaging with forward-directed observation direction.

Identical parts are labeled with the same reference signs in all the figures.

### DETAILED DESCRIPTION OF THE INVENTION

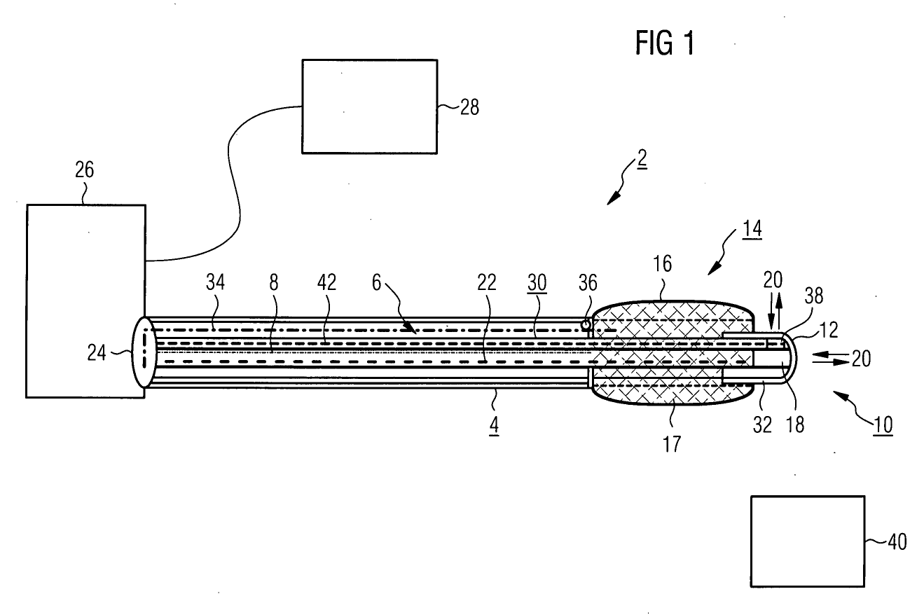

The catheter device 2 shown in FIG. 1 is designed to allow a minimally invasive intervention in a cardiac valve. It comprises a flexible catheter sheath 4 for introducing into a blood vessel which is not shown in further detail. The catheter sheath 4 encloses a cylindrical catheter cavity 6 (also referred to as a lumen) in which runs a control line 8 (only indicated schematically) for controlling a catheter-specific module 14 disposed at the proximal end 10 in the region of the catheter tip 12. The catheter-specific module 14 comprises means (not shown in further detail here) for transporting and precisely positioning an artificial cardiac valve 16 which is to be implanted and which, in the transport state, is advantageously folded, by means of a fixing stent 17. The approximately cylindrical wire or plastic mesh structure of the fixing stent 17 is shown only schematically in FIG. 1; the valvular cusps are not visible. Alternatively or in addition, the module 14 can comprise surgical tools which can be operated via the control line 8 (e.g. a surgical scalpel, gripping and holding means, etc.) for an annuloplastic intervention in a cardiac valve.

In order to ensure an optimally successful and permanent recovery and to minimize any intervention risks it is important for the catheter-specific module 14 by means of which the intervention is performed locally to be positioned as accurately as possible at the correct or “appropriate” point in the heart for the respective intervention, which in the prior art was typically achieved by means of angiographic X-ray monitoring. For an improved monitoring of the catheter's advance and a more precise placement of the catheter-specific module 14 even without use of ionizing X-ray radiation, the catheter device 2 according to FIG. 1 is equipped with an imaging sensor 18 in the region of the catheter tip 12. Depending on sensor type and other details of the embodiment, its “field of vision” is preferably directed radially outward (toward the surrounding vascular wall, not shown here) and/or forward in the proximal direction (in other words, in the feed direction of the catheter device 2), as indicated symbolically by means of the arrows 20.

The imaging sensor 18 can be, for example, an optical sensor, an acoustic (ultrasound) sensor or a sensor based on the magnetic resonance principle. The signal and supply lines 22 necessary for its operation and for transmitting the recorded image data are routed in the interior of the catheter sheath 4 up to a connection coupling 24 disposed at the distal end (facing away from the body) of the catheter device 2. Firstly, the pressure- and/or fluid-conducting lines inside the catheter sheath 4 can be mechanically connected to external storage containers and the like via the connection coupling 24. Secondly, the electronic imaging components of the catheter device 2 can be electrically connected to a signal interface 26 (only indicated schematically) via the connection coupling 24, said signal interface 26 in turn being connected to an external image processing and playback apparatus 28. A monitor (not shown in further detail) serves to play back the “live images” of the treatment site recorded intravascularly or, as the case may be, intracorporally by the imaging sensor 18 and if necessary subsequently computationally edited.

In order to be able to rotate the imaging sensor 18 about its own axis inside the stationary catheter sheath 4, a rotatable driveshaft can also be disposed in the catheter cavity 6, though said driveshaft is not shown in further detail in FIG. 1. The imaging sensor 18, the signal lines 22 and where appropriate the driveshaft can be combined into a compact unit in the manner of an inner catheter disposed inside the outer catheter sheath 4 and surrounded by an (inner) protective sheath 30. In particular when interferometric imaging methods are used, fiber-optic light guides can also be routed in the inner catheter and incident and emergent light beams can be guided via said light guides to an externally installed interferometer unit or the like which can be connected via the connection coupling 24. In the region of the imaging sensor 18 the inner protective sheath 30, if necessary also the outer catheter sheath 4, advantageously has a transparent window 32, possibly also an optical lens, for the respective imaging method.

Furthermore, one or more lines 34 can (optionally) be provided for a flushing fluid or a contrast agent which can be injected into the vascular or cardiac valve region that is to be examined/treated via an outlet aperture 36 disposed close to the catheter-specific module 14.

Finally, position sensors 38 can be provided in the region of the catheter tip 12, here in FIG. 1 in immediate proximity to the imaging sensor 18, which position sensors 38 operating on the transmitter-receiver principle in interaction with a position detection unit 40 disposed outside the patient's body enable a precise pinpointing/localization of the catheter tip 12 by identifying the coordinates of the catheter tip. The position data thus acquired can be supplied to the image processing and playback apparatus 28, for example, and taken into account during the image reconstruction, specifically for artifact correction. The necessary signal lines 42 for the position sensors 38 can also be routed inside the (inner) protective sheath 30 essentially in parallel with the signal lines 22 of the imaging sensor 18.

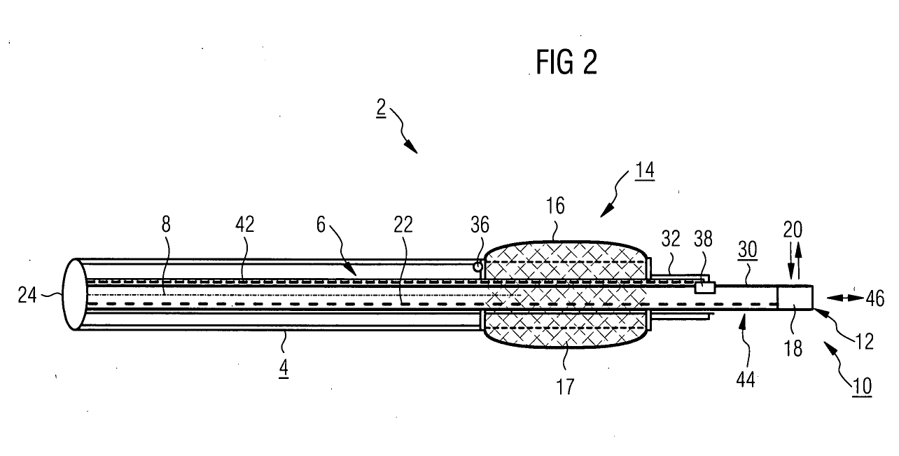

Structural modifications of the catheter device 2 are shown in each case in FIG. 2 to FIG. 6.

Thus, for example, in FIG. 2 the internal part 44 carrying the imaging sensor 18 can be displaced relative to the catheter sheath 4 in a forward (proximal) direction from a retracted position (not identified more precisely) corresponding to the position in FIG. 1 into an advanced position, and vice versa (indicated by the double arrow 46). This means that the imaging sensor 18 can be pushed forward if necessary beyond the region of the catheter tip 12 with the transparent window 32 and at that position has an totally unrestricted view. A transparent window can then possibly be dispensed with entirely, as shown in FIG. 3.

The embodiment according to FIG. 4 essentially corresponds to that from FIG. 1, although in this variant the position sensor(s) 38 is (are) now disposed on the outer catheter sheath 4. In the variant according to FIG. 5, finally, the displacement path of the imaging sensor 18 is increased in size in the longitudinal direction and a transparent window 32 with a greater spatial extension is provided accordingly. In this case the position sensors 38 are likewise mounted further toward the end of the catheter device 2 facing away from the body, at the rear end of the catheter-specific module 14.

In the variant according to FIG. 6 an additional dilatation balloon 47 is disposed ahead of the module 14 toward the proximal end 10, said balloon being expandable by way of an expansion means which can be supplied via an expansion means line (not shown here) running inside the catheter sheath 4. In this way the diseased cardiac valve region can be dilated prior to the positioning and decoupling of the artificial cardiac valve 16.

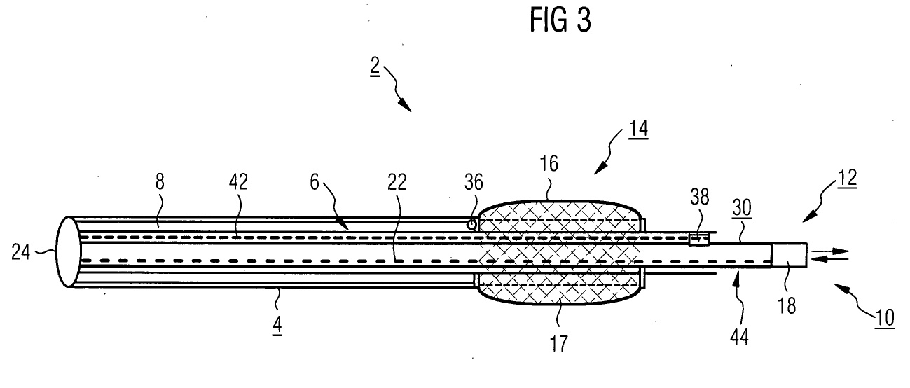

In the detail view according to FIG. 7 the region of the catheter tip 12 with the imaging sensor 18 is enlarged and highlighted, a CMOS-based optical sensor being used in the variant shown here. A light source 48, in this case a high-performance micro-LED, illuminates the vascular wall 50 surrounding the catheter device 2 and specifically the imaging sensor 18 in a roughly annular shape (emitted light 51). Light 53 reflected off the vascular wall 50 falls through a lens 52 onto a reflection mirror 54 (or also e.g. onto a prism with an analogous operating principle or beam guidance) and from there onto the actual CMOS image detector 56. The arrangement according to FIG. 7 is therefore configured for a radial line of sight (referred to the central axis 58 of the catheter device 2). The full lateral 360° field of vision can be covered by means of a rotational movement around the central axis 58, indicated by the arrow 60, effected with the aid of the driveshaft 59.

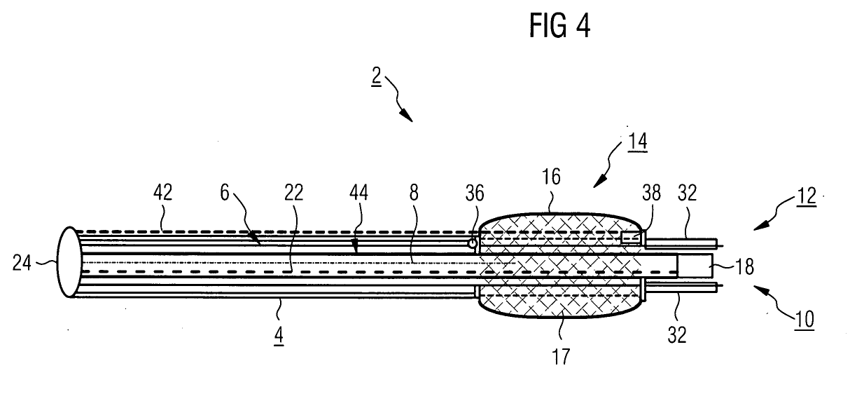

Alternatively, FIG. 8 shows an example of a configuration of light source 48, lens 52 and CMOS detector 56 by means of which a forward-directed observation is made possible which is particularly useful during the advancing of the catheter device 2 through the blood vessels to the heart ventricles and if necessary through the cardiac valves. An obstacle 61 lying in the forward direction and possibly obstructing the further advance can be detected in this way. The two variants according to FIG. 7 and FIG. 8 can, if necessary, also be combined with each other in order to provide a particularly comprehensive field of vision in practically all directions.

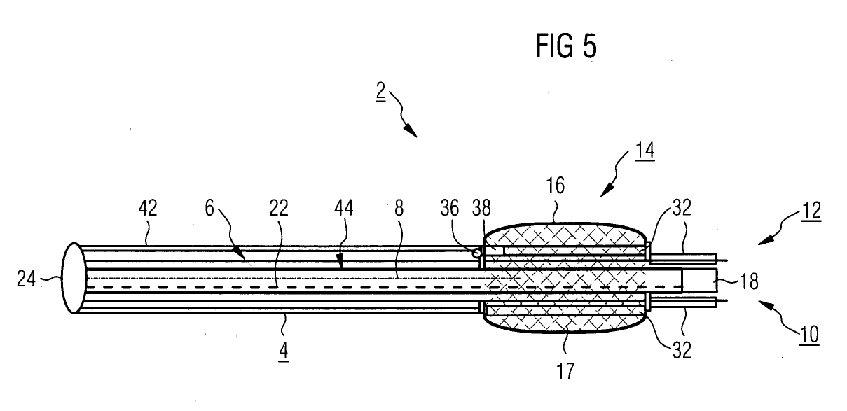

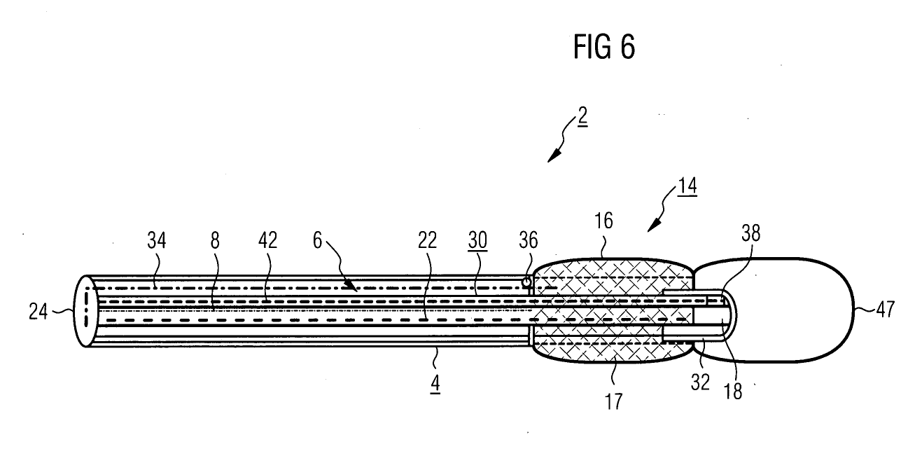

The above-mentioned observation directions, namely radial/lateral and forward-directed, can also be realized with other sensor types. For example, a configuration of an OCT or LCI sensor head 62 for radial radiation and reception is shown in FIG. 9, and a similar configuration for forward-directed radiation and reception is shown in FIG. 10. More precisely, the reference sign 62 denotes only the sensor part or sensor head responsible for coupling the light into and out of the fiber-optic light guide 64; the actual interferometric analysis and image generation takes place outside the catheter device 2. Shown in each case is the optical path of decoupled and reflected light beams influenced by the reflection mirror 66 and the lens 68.

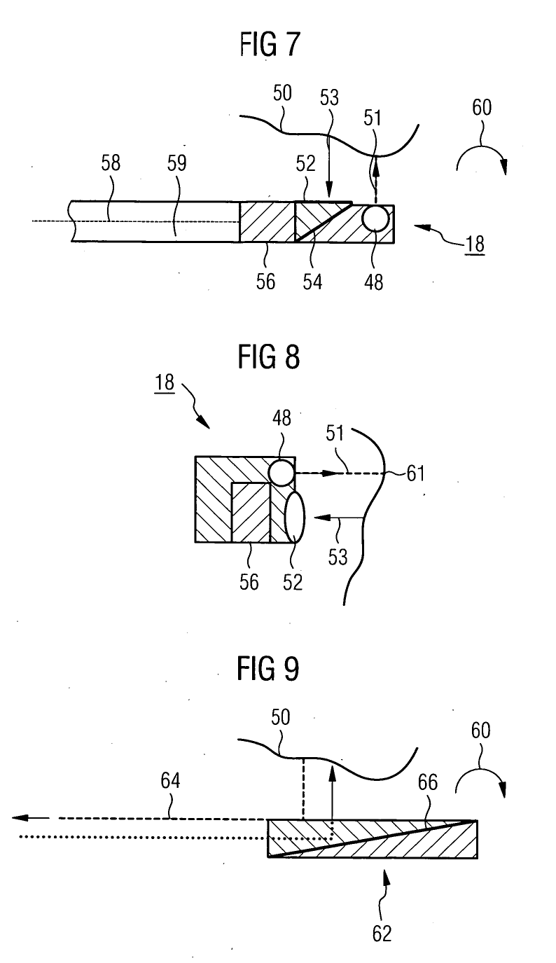

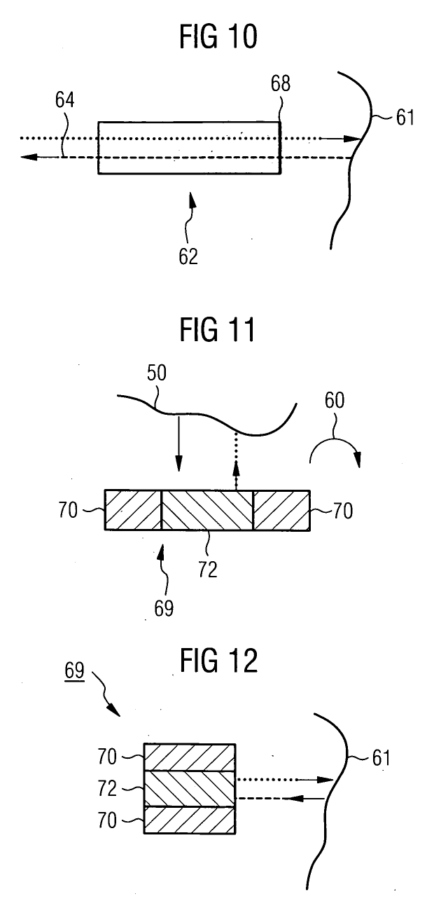

In similar fashion an IVMRI sensor or IVUS sensor can also be configured either for radial or for forward-directed radiation/reception, as shown schematically in FIG. 11 and FIG. 12 for an IVMRI sensor 69 with permanent magnet 70 for the static magnetic field and transmitting/receiving coils 72.

With lateral radiation/reception, instead of providing a single rotating sensor it can be advantageous, in particular in the case of ultrasound sensors, to provide an array of ultrasound sensor elements with different “viewing directions” which are activated, i.e. excited and polled, for example cyclically via a multiplexer.

1.-13. (canceled)

14. A catheter device for treating a cardiac valve disease, comprising:
a catheter cavity;
a flexible catheter sheath that encloses the catheter cavity;
a catheter-specific module disposed at a proximal end close to a catheter tip that transports and positions a cardiac valve implant; and
an image sensor arranged in a region of the catheter tip that records an image of the treatment,
wherein the image sensor is configured to be mounted longitudinally relative to the catheter sheath so that it can be moved into an advanced position outside of the catheter sheath.

15. The catheter device as claimed in claim 14, wherein the image sensor is aligned so that a field of vision of the image sensor covers a spatial region around the catheter-specific module.

16. The catheter device as claimed in claim 14, wherein the image sensor is aligned so that a field of vision of the image sensor covers a spatial region lying in front of the catheter tip.

17. The catheter device as claimed in claim 14, wherein the imaging sensor is an optical image sensor.

18. The catheter device as claimed in claim 14, wherein the imaging sensor is a magnetic resonance element.

19. The catheter device as claimed in claim 14, wherein the imaging sensor is an ultrasound element.

20. The catheter device as claimed in claim 14, wherein the imaging sensor rotates relative to the catheter sheath via a driveshaft routed in the catheter cavity.

21. The catheter device as claimed in claim 14, wherein a plurality of imaging sensors are distributed over a circumference of the catheter sheath and arranged stationary relative to the catheter sheath.

22. The catheter device as claimed in claim 21, wherein data from the imaging sensors is cyclically readout via a multiplexer.

23. The catheter device as claimed in claim 14, wherein the catheter-specific module surgically models the cardiac valve.

24. A medical examination and treatment apparatus having a catheter device for treating a cardiac valve disease, comprising:
a catheter cavity;
a flexible catheter sheath that encloses the catheter cavity;
a catheter-specific module disposed at a proximal end close to a catheter tip that transports and positions a cardiac valve implant; and
an image sensor arranged in a region of the catheter tip that records an image of the treatment;
a signal line that is routed in the catheter cavity;
an image processing and playback apparatus that is located outside the catheter device and connected with the image sensor via the signal line,
wherein the imaging sensor is configured to:
be mounted longitudinally relative to the catheter sheath so that it can be moved into an advanced position outside of the catheter sheath, and
transmit the image from a location of the treatment to the image processing and playback apparatus in a real time.

25. A method for performing a minimally invasive intervention examination in a cardiac valve by a catheter device having a catheter-specific module, comprising:
transporting and positioning a cardiac valve implant by the catheter-specific module;
integrating an imaging sensor into a proximal end of the catheter device;
guiding the imaging sensor through a blood vessel of a patient up to the cardiac valve;
recording a real time image of the intervention by the image sensor;
transmitting the real time image to a playback unit located outside a body of the patient; and
monitoring an advance of the catheter device and a position of the catheter-specific module based on the transmitted real time image.

26. The method as claimed in claim 25, wherein the catheter-specific module surgically models the cardiac valve.

27. The method as claimed in claim 26, wherein the positioning of the cardiac valve implant or the surgical modeling of the cardiac valve is monitored based on the transmitted real time image.

28. The method as claimed in claim 26, further comprising:
positioning the patient on a treatment table;
preparing the examination;
introducing the catheter device via a venous access port;
guiding the catheter device based on the transmitted real time image up to the cardiac valve;
simultaneously transporting the cardiac valve implant arranged on the catheter-specific module;
monitoring the cardiac valve by the transmitted real time image;
positioning the catheter device based on the transmitted real time image;
monitoring the positioning of the cardiac valve implant or the surgical modeling of the cardiac valve in real time by the transmitted real time image;
concluding the examination by double-checking the transmitted real time image; and
transferring the patient.

29. The method as claimed in claim 25, wherein the examination is an X-ray examination or an extracorporal ultrasound examination.
